(19) United States
(12) Patent Application Publication
(10) Pub. No.: US 2006/0064021 A1
(43) Pub. Date: Mar. 23, 2006

(54) Detection and correction of catheter line distortion in blood pressure measurements

(76) Inventor: David Hefele, Winter Springs, FL (US)

Correspondence Address: QUARLES & BRADY LLP, 411 E. WISCONSIN AVENUE, SUITE 2040, MILWAUKEE, WI, 53202-4497, US

(21) Appl. No.: 10/945,381

(22) Filed: Sep. 20, 2004

(51) Int. Cl. A61B 5/02

(52) U.S. Cl. 600/486

(57) ABSTRACT

An invasive blood pressure monitor assesses likelihood of coloration of the pressure waveform by the fluid-filled catheter prior to imposing a correction on this pressure waveform. This detection may be made by comparing two alternate signal processing paths of the pressure waveform, one of which is intended to correct for coloration and applying the coloration correction only if those processing paths yield significantly different output values.

### CROSS-REFERENCE TO RELATED APPLICATIONS

--

### STATEMENT REGARDING FEDERALLY SPONSORED RESEARCH OR DEVELOPMENT

--

### BACKGROUND OF THE INVENTION

The present invention relates generally to invasive blood pressure measuring equipment and in particular to a simple and robust circuit for detecting and correcting measurement errors caused by catheter line resonance.

High accuracy blood pressure measurements may use a catheter communicating directly between a patient's artery and an external pressure transducer. Normally the catheter is wholly or partially filled with saline solution to provide a continuous liquid path between the artery and pressure transducer. The mass of fluid in the catheter and the inherent elasticity of the catheter can introduce a distortion to the pressure readings obtained. Of particular concern is resonance which may accentuate harmonics of the cardiac rhythm to undesirably alter systolic or diastolic pressure readings.

A number of techniques have been used to compensate for this distortion. Mechanical damping may be introduced into the fluid circuit, for example, in the form of a restriction in the catheter to attenuate any resonance. Analogously, low-pass filtering of the pressure signal, using an electrical circuit receiving a signal from the pressure transducer, may be used to attenuate these harmonics. If the physical characteristics of the catheter are fully known, the catheter's effect on the blood pressure signal may be modeled and sophisticated inverse transform techniques may be used to eliminate or reduce this distortion.

Typically the monitoring instrument that receives the electrical signal from the pressure transducer must work with a variety of different catheters having different characteristics and lengths, the latter possibly altered by the physician to meet the demands of the situation. This variation in catheters places a practical limit on the ability to employ reverse modeling algorithms. Catheter indifferent techniques, such as those which attenuate the harmonics either mechanically or electrically, introduce their own “coloring” to the blood pressure signal and particularly in the case where the harmonics are low, can make blood pressure data less accurate than it would have been without such compensation.

What is needed is a simple and robust method of correcting for distortion of blood pressure readings by the catheter that works with a variety of catheters and that does not unnecessarily degrade the blood pressure signal.

### BRIEF SUMMARY OF THE INVENTION

The present invention provides a simple circuit that suppresses resonance-induced harmonics only after determining that there is a probability of resonance induced distortion. In this way, when low or non-resonant catheter lines are employed, the blood pressure waveform may be detected directly and the possible distortions of any correction system avoided.

Specifically then, the present invention provides a blood pressure monitor for use with an invasive catheter system having a first and second signal processor receiving an electrical pressure signal from the catheter and providing different degrees of attenuation of resonance components of the electrical pressure signal. A comparison circuit monitors a divergence between the outputs of the first and second signal processors and based on that divergence selects one output as a corrected pressure signal. A display outputs at least one blood pressure measurement to an operator based on the corrected pressure output.

Thus is it one object of at least one embodiment of the invention to tie any correction of blood pressure to an assessment of whether significant correction is required. In this way unnecessary distortion induced by the correction process itself is minimized.

The signal processors may be low-pass filters with a first filter having a lower cutoff frequency than a second filter.

Thus is it one object of at least one embodiment of the invention to provide a simple and well-characterized correction process.

The first filter may have a cutoff frequency between a second and third harmonic of a standard blood pressure signal.

It is thus another object of at least one embodiment of the invention to provide a correction system that leaves intact the major spectral components of the blood pressure signal.

The blood pressure monitor may include a pulse rate monitor providing a pulse rate output and the first filter may receive the pulse rate output to change the cutoff frequency as a function of the pulse rate output.

Thus it is an object of at least one embodiment of the invention to adapt to a variety of different pulse rates.

The second cutoff filter may have a frequency above a fifth harmonic of a standard blood pressure signal.

Thus it is one object of at least one embodiment of the invention to provide one signal path that essentially passes the blood pressure signal without significant distortion to provide the fidelity measurement when no catheter resonance is suspected.

The comparison circuit, or computer algorithm, may select one output of one signal processor as a function of the divergence between outputs at a pre-determined phase of the blood pressure, for example, the peak blood pressure.

Thus it is another object of at least one embodiment of the invention to provide a simple method of determining if distortion exists. The sharp portion of the peak blood pressure is believed to be particularly susceptible to distortion by harmonics.

The divergence may be a pre-determined pressure difference between outputs.

Thus is it one object of at least one embodiment of the invention to provide a simple mathematical process to detect distortion comparing the output of the existing signal processors.

The signal processors may be implemented in a computer in software, for example, the low-pass filters may be implemented by an averaging of successive data samples.

It is thus another object of at least one embodiment of the invention to provide signal processing that is well behaved mathematically and that requires relatively little computer processing resource.

These particular objects and advantages may apply to only some embodiments falling within the claims and thus do not define the scope of the invention.

### BRIEF DESCRIPTION OF THE DRAWINGS

FIG. 1 is a simplified perspective view of an invasive blood pressure monitor employing a catheter to connect a pressure transducer to blood in an artery for direct blood pressure measurement;

FIG. 2 is a plot of blood pressure versus time for actual arterial pressure and for a pressure transducer signal colored by resonance of the catheter line;

FIG. 3 is a block diagram of the present invention showing two signal processors whose outputs are compared for detection of signal coloring; and

FIG. 4 is a spectrum of the harmonics of the blood pressure waveform of FIG. 2 showing placement of cutoff frequencies of the low-pass filters for one embodiment of the signal processors of FIG. 3.

### DETAILED DESCRIPTION OF THE PREFERRED EMBODIMENT

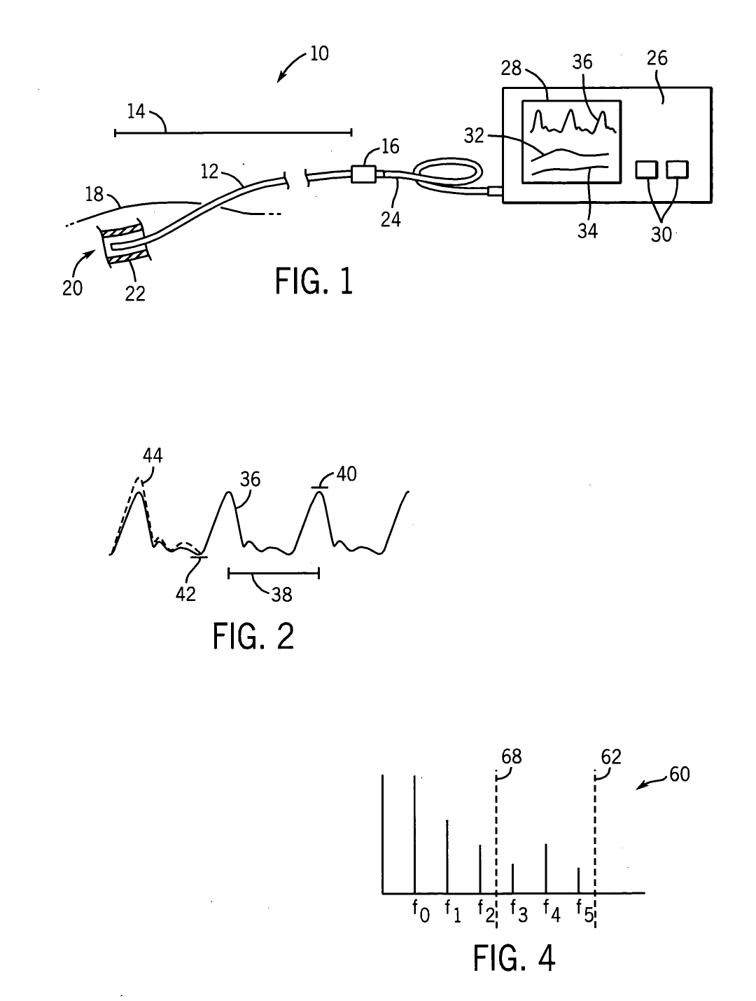

Referring now to FIG. 1, a blood pressure monitoring system 10 for use in invasive blood pressure monitoring may include a catheter 12 having an approximate length 14 and extending between a pressure transducer 16 outside of a patient 18 and a pressure monitoring point 20, for example, located within a patient artery 22.

The catheter 12 is filled with a saline solution and may include pressure equalization and saline introduction ports (not shown) as would be understood to those of ordinary skill in the art.

The pressure transducer 16 provides an electrical signal proportional to a pressure of the liquid in the catheter 12 at the pressure transducer 16. This electrical signal may be communicated by a conductive cable 24 to a monitoring unit 26, the latter which includes a display 28 and user controls 30. The display may show an actual pressure waveform 36 and/or numerical values for the systolic pressure, diastolic pressure, or mean pressure based on the electrical signal from the pressure transducer 16 as is generally understood in the art.

Referring now to FIG. 2, an actual pressure waveform 36 reflecting blood pressure at pressure monitoring point 20 may have a repeating pattern with a period 38 corresponding to the pulse rate of the patient and having a peak 40 providing an instantaneous systolic pressure and a trough 42 providing an instantaneous diastolic pressure.

As discussed above, resonances and other distortion caused by the physical quality of the catheter 12 may produce a distorted pressure waveform 44 from the pressure transducer 16, in this case, providing an artificially high systolic pressure as the result of a constructive adding of the actual pressure waveform 36 and one or more resonance harmonics. This distortion may be a complex function of the spectral characteristic of the actual pressure waveform 36 and is not a simple scaling that can be corrected by standard calibration.

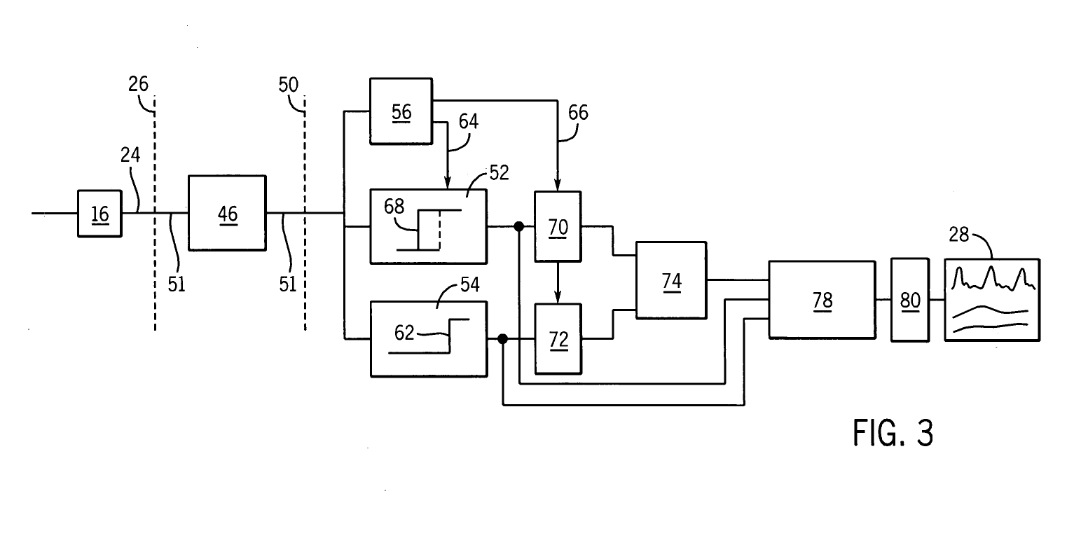

Referring now to FIG. 3, the measured blood pressure 51 on cable 24 from the pressure transducer 16 may have little distortion, for example, when the catheter 12 is short, and thus resemble actual pressure waveform 36 or may have significant distortion per distorted pressure waveform 44. The measured blood pressure 51 is received by input circuitry 46 providing generally input amplifiers, a sampling circuit, and an analog to digital converter of types well known in the art. The input circuitry 46 converts the electrical signal to a series of digital samples that may be read, stored, and processed by a microcontroller or processor 50. The digital samples will accurately reflect the measured blood pressure 51, and thus will also be treated as measured blood pressure 51.

In the preferred embodiment, the processor 50 implements a number of processing blocks that will now be described. It will be understood to one of ordinary skill in the art that these processing blocks may also be implemented through discreet circuitry according to well-known techniques or by a combination of hardware and software.

After being received by the processor 50, the measured blood pressure 51 is simultaneously processed by a first signal processor 52 and a second signal processor 54 in parallel and by a pulse rate detector 56 which will be described in more detail below.

The pulse rate detector 56 may use any of the number of well known programs employing, for example, thresholds intended to identify the peaks 40 and troughs 42 with reference to a running average so as to accommodate slowly varying baseline changes. The pulse rate detector 56 provides a pulse rate output 64, for example, 90 beats per minute or 120 beats per minute and a phase output 66 indicating a particular point in the cardiac phase, for example, a peak 40 or trough 42.

Referring now to FIG. 4, the spectrum 60 of the measured blood pressure 51 will include a fundamental f₀ representing the particular pulse rate of the individual together with significant spectral components at a first harmonic f₁ and second harmonic f₂ together with additional harmonics of higher order. As will be understood to those of ordinary skill in the art, the first harmonic f₁ is of twice the frequency of f₀, the second harmonic at three times the frequency of f₀, and so forth.

Referring now to FIGS. 3 and 4, the second signal processor 54 implements a low-pass filter having a cutoff frequency 62 positioned somewhere above the fifth harmonic f₅. This filter is intended to pass the measured blood pressure 51 waveform without significant modification while removing noise and the cutoff frequency 62 is set based on the empirical observation that most of the spectral energy of a typical actual blood pressure waveform 36 is concentrated below the fifth harmonic f₅.

Thus, in the absence of significant coloration by the catheter 12, a high fidelity actual blood pressure waveform 36 will pass unmodified through the second signal processor 54.

Referring still to FIGS. 3 and 4, the first signal processor 52 implements a low-pass filter having a variable cutoff frequency 68 positioned using pulse rate output 64 to vary it between the second harmonic f₂ and the third harmonic f₃. In this way as the heart rate increases, the cutoff frequency 68 may increase proportionally to remain in fixed relationship with these harmonics. Thus, for example, at a heart rate of 90 beats per minute, the cutoff frequency 68 may be set to 6 Hertz, however, if the patient's pulse rate is 120 beats per minute, then the cutoff frequency may be set to 7.5 Hertz.

These filters may be readily implemented through a number of well-known computer algorithms including those which average waveforms over a pre-defined window either once or doubly to provide the necessary filtration characteristics. Such filtration represented by averaging is well characterized and thus would be unexpected to produce significant signal artifacts over the wide range of distorted pressure waveform 44. Changing of this cutoff frequency 68 is easily effected through software by, for example, changing the window of the average.

Referring again to FIG. 3, the outputs of the first signal processor 52 and the second signal processor 54 are captured by corresponding sample and hold circuits 70 and 72, respectively. In the preferred embodiment, this sampling occurs at the peak 40 of the measured blood pressure 51 thus capturing a systolic pressure.

The systolic pressures from sample and hold circuits 70 and 72 are received by a comparator 74 which operates to detect possible corruption of the measured blood pressure 51 by catheter resonance as deduced by a difference between the outputs of sample and hold circuits 70 and 72 of more than a pressure difference of 5 millimeters of mercury. If the outputs of sample and hold circuits 70 and 72 are within 5 millimeters of mercury, then it is inferred that there is no significant coloration of the measured blood pressure 51 by the catheter 12 and the output from second signal processor 54, having only minimal high frequency suppression, is passed directly to display circuitry 80 by router 78 receiving an output from the comparator 74 and the outputs of the first signal processor 52 and second signal processor 54. This output is passed to display 28 to provide both a pressure waveform 36 and systolic plots 32 and diastolic plots 34.

On the other hand, if the difference between the outputs of waveform sample and hold circuits 70 and 72 exceed the threshold of a pressure of 5 millimeters of mercury, then the output from first signal processor 52 is provided by router 78 to display circuitry 80.

It will be understood that the threshold of 5 millimeters of mercury may be varied according to empirical refinement.

The present invention thus not only provides an extremely simple and well-characterized correction of possible coloration of the waveform 36, but also limits the correction to provide a direct readout in those instances where more accurate data is likely to be obtained without correction.

It is specifically intended that the present invention not be limited to the embodiments and illustrations contained herein, but include modified forms of those embodiments including portions of the embodiments and combinations of elements of different embodiments as come within the scope of the following claims.

We claim:

1. A blood pressure monitor for use with an invasive catheter system employing a fluid filled catheter having a first end communicating with a blood vessel of a patient and a second end received by a pressure transducer producing an electrical pressure signal indicating fluid pressure at the second end, the blood pressure monitor comprising:
a first and second signal processor receiving the electrical pressure signal and providing different degrees of attenuation of predetermined resonance components of the electrical pressure signal;
a comparison circuit monitoring a divergence between outputs of the first and second signal processors and based on that divergence selecting one output as a corrected pressure output; and
a display outputting at least one blood pressure measurement to an operator based on the corrected pressure output.

2. The blood pressure monitor of claim 1 wherein the first signal processor provides more attenuation of resonance components than the second signal processor and wherein the output of corrected pressure output is selected to be the output of the first signal processor when the divergence of the first and second signal processor is beyond a predetermined amount and otherwise to be the output of the second signal processor.

3. The blood pressure monitor of claim 1 wherein the first and second signal processors are lowpass filters wherein the first filter has a lower cutoff frequency than the second filter.

4. The blood pressure monitor of claim 3 wherein the first filter has a cutoff frequency between the second and third harmonic of a standard blood pressure signal.

5. The blood pressure monitor of claim 3 further including a pulse rate monitor providing a pulse rate output and wherein the first filter receives the pulse rate output to change the cutoff frequency as a function of pulse rate output.

6. The blood pressure monitor of claim 5 wherein the first filter has a cutoff frequency between a second and third harmonic of a standard blood pressure signal.

7. The blood pressure monitor of claim 3 wherein the second filter has a cutoff frequency above a fifth harmonic of a standard blood pressure signal.

8. The blood pressure monitor of claim 1 wherein the comparison circuit selects one output as a function of the divergence between outputs at a predetermined phase of a blood pressure.

9. The blood pressure monitor of claim 8 wherein the predetermined phase is a peak blood pressure.

10. The blood pressure monitor of claim 1 wherein the divergence is a predetermined pressure difference between outputs.

11. The blood pressure monitor of claim 1 wherein the signal processors are implemented in a computer in software.

12. The blood pressure monitor of claim 11 wherein the signal processors are low-pass filters implemented by an averaging of successive data samples.

13. A blood pressure monitor for use with an invasive catheter system employing a fluid filled catheter having a first end communicating with a blood vessel of a patient and a second end received by a pressure transducer producing an electrical pressure signal indicating fluid pressure at the second end, the blood pressure monitor comprising:
a distortion detection circuit monitoring the electrical pressure signal to detect a likelihood of catheter induced distortion caused by mechanical resonance of the catheter system;
a correction filter processing the electrical pressure signal to reduce resonance harmonics only when the distortion detection circuit detects a likelihood of catheter induced distortion; and
a display outputting at least one blood pressure measurement to an operator based on the output of the correction filter.

14. A method of evaluating blood pressure measurements obtained with an invasive catheter system employing a fluid filled catheter having a first end communicating with a blood vessel of a patient and a second end received by a pressure transducer producing an electrical pressure signal indicating fluid pressure at the second end, the method comprising the steps of:
(a) filtering the electrical pressure signal with a first and second filter attenuating predetermined frequency components of the electrical pressure signal;
(b) comparing a divergence between outputs of the first and second filters and, based on that divergence, selecting one output as a corrected pressure output; and
(c) outputting at least one blood pressure measurement to an operator based on the corrected pressure output.

15. The method of claim 14 wherein the first filter provides more attenuation of harmonics than the second filter and wherein the output of corrected pressure output is selected to be the output of the first filter when the divergence of the first and second filters is beyond a predetermined amount and otherwise to be the output of the second filter.

16. The method of claim 14 wherein the first and second filters are lowpass filters wherein the first filter has a lower cutoff frequency that the second filter.

17. The method of claim 16 wherein the first filter has a cutoff frequency between the second and third harmonic of a standard blood pressure signal.

18. The method of claim 16 including the step of adjusting the cutoff frequency of the first filter as a function of pulse rate output.

19. The method of claim 18 wherein the first filter has a cutoff frequency between the second and third harmonic of a standard blood pressure signal.

20. The method of claim 16 wherein the second filter has a cutoff frequency above a fifth harmonic of a standard blood pressure signal.

21. The method of claim 14 wherein the comparison step is performed at a predetermined phase of a blood pressure.

22. The method of claim 21 wherein the predetermined phase is a peak blood pressure.

23. The method of claim 14 wherein the divergence is a predetermined pressure difference between outputs.
